(19) United States
(12) Patent Application Publication
(10) Pub. No.: US 2016/0174833 A1
(43) Pub. Date: Jun. 23, 2016

(54) EXAMINING SYSTEM FOR EYE GLAND FUNCTION

(71) Applicants: UNITED INTEGRATED SERVICES CO., LTD., TAIPEI CITY (TW); NATIONAL YANG-MING UNIVERSITY, TAIPEI CITY (TW)

(72) Inventors: HUIHUA CHIANG, TAIPEI CITY (TW); O. CHANG, NEW TAIPEI CITY (TW); SHU-WEN CHANG, TAIPEI CITY (TW); CHUNG-HWA CHANG, NEW TAIPEI CITY (TW); TAI-YUAN SU, NEW TAIPEI CITY (TW); CHIEN-YI LU, TAIPEI CITY (TW)

(21) Appl. No.: 14/620,806

(22) Filed: Feb. 12, 2015

(30) Foreign Application Priority Data
Dec. 19, 2014 (TW) 103144526

(51) Int. Cl. A61B 3/10; A61B 5/01

(52) U.S. Cl. CPC A61B 3/101; A61B 5/015

(57) ABSTRACT

The disclosure is related to an examining system for eye gland function. The system includes a thermography unit used to capture an ocular thermographic image. The temperature distribution over multiple positions of eye can be found. A computation unit is used to compute mean temperatures for the areas of multiple positions. The temperatures are such as a reference temperature for a reference position and temperatures for multiple tested positions. The temperature values are temporarily stored in a memory of the system. The system further includes a comparison unit used to calculate the differences or ratios among the temperature values. The comparison results are output. According to one embodiment, the reference temperature may be a mean temperature within an area of an upper palpebral; and the temperatures for the multiple tested positions may be the mean temperatures of multiples positions along a line from left to right over the eyelid.

### BACKGROUND

1. Technical Field

The present invention is generally related to a system for examining eye gland function, in particular to an examining system using thermography to capture a thermal image of an eyelid for examining the eye gland function.

2. Description of Related Art

Diagnosis of eye disease usually refers to visual inspection, stereoscopic perception examination, intraocular pressure check, or tears examination. For examining dry eye syndrome, a kind of examination of tears secretion or quality of tears is introduced. The problems of disorder of tears balance or deficiency of tears may result in the dry eye syndrome.

However, the crucial point causing the mentioned problems is in regards to the meibomian glands. The meibomian glands are a kind of eye glands around the eyelid. One of the major objectives of the meibomian glands is to secrete the lipid layer of the tears. It is noted that there are three layers within a tear film. The main components within the tear film include such as an aqueous layer secreted by the lachrymal gland. A mucous layer covering the cornea surface is also provided for latching the aqueous layer. Further, the lipid layer secreted by the meibomian glands is included. The lipid layer covers the aqueous layer for the function of preventing the aqueous layer from rapid evaporation. The lipid layer also enhances surface tension of the tear film, and lubricates the contact surface between the eyelid and the eyeball.

The major disability of the meibomian glands is due to the obstruction of the meibomian glands, or an unusual secreted mass of oil and fat. The disability of the meibomian glands downgrades the quality of oil and fat, and reduces stability of the tear film. The disability of the meibomian glands speeds up the evaporation of tears, and easily results in dry eye syndrome because the meibomian glands get an inflammation.

Dry eye syndrome occurs if the eyes do not produce enough tears or the tears evaporate too quickly. To examine the dry eye syndrome, according to conventional technology, is to verify if the tear film is broken. The examination may be roughly divided into a contact and contactless type of approach. The contact type of approach is such as Schirmer's test which determines if the eye produces enough tears to keep the eye moist. Schirmer's test uses a filter paper contact with the eye for several minutes to check if any dry eye syndrome symptom appears from the diffusion of tears. A scheme of Tear Break Up Time (TBUT) is another method to observe the first broken time of the tear film by inserting fluorescein dye to the eye of patient. The symptom of dry eye syndrome appears if the tear film is broken too early.

The contactless approach allows an ophthalmologist to observe the condition of the tear film of the patient by his human eyes. Further, an infrared thermal detector may be used to measure the temperatures over an ocular surface for inferring with the broken condition of the tear film. However, the kinds of contactless approaches rely on the ophthalmologist's judgment through clinical interview. There is no quantitative standard for the diagnosis since different doctors may provide various explanations of the broken tear film. The conventional technologies may have high probability of misjudgment.

### SUMMARY

The disclosure is related to a non-contact examining system for the human eye gland, for example the lachrymal gland. Thermography may be incorporated to measure ocular temperature distribution. The temperature information over the eye gland is a basis to examine function of the eye gland. The embodiments in the disclosure are directed to the examining system for the eye gland.

In the examining system, a thermography unit used to acquire an ocular thermographic image is included. The thermography unit may be a standalone device, or a sensor circuit integrated into the system. The thermography unit aims at acquiring temperature distribution of multiple positions of the ocular thermographic image. The system further includes a computation unit operated to measure a mean temperature for the multiple positions within an area. At least a reference temperature of a reference position is obtained. The reference temperature is stored into a first temperature retrieval unit. The temperatures of the multiple tested positions may be stored into a second temperature retrieval unit. The system includes a comparison unit. After acquiring the reference temperature and the temperatures of tested positions, the comparison unit is used to calculate temperature differences or ratios among the reference temperature and the temperatures of the tested positions. The system further includes an output unit for outputting the result made by the comparison unit.

The examining system may incorporate a positioning unit to position a plurality of positions on the ocular thermographic image. For example, the position of an eyelid and multiple tested positions from left to right of the eyelid are defined. The temperatures of these positions are obtained. When the temperatures of the positions are acquired, differences or ratios can be obtained by comparing the temperatures of the positions with the reference temperature. The differences or ratios are employed to diagnose an index of the functional disturbance of eye gland.

In order to further understand the techniques, means and effects of the present disclosure, the following detailed descriptions and appended drawings are hereby referred to, such that, and through which, the purposes, features and aspects of the present disclosure can be thoroughly and concretely appreciated; however, the appended drawings are merely provided for reference and illustration, without any intention to be used for limiting the present disclosure.

### BRIEF DESCRIPTION OF THE DRAWINGS

FIG. 1 shows thermography adopted in the examining system for an eye gland according to one embodiment of the present invention;

FIG. 2 shows a diagram schematically describing the temperature-rise curve of blackbody;

FIG. 3 shows computation circuit or software module implementing the examining system in one embodiment of the present invention;

FIG. 4 shows computation circuit or software module implementing the examining system in one further embodiment of the present invention;

FIG. 5 shows a diagram depicting the system for examining the tested portion of closed eye according to the present invention;

FIG. 6 shows a flow chart illustrating a method for examining the eye gland in one embodiment in accordance with the present invention;

FIG. 7 shows another flow chart illustrating the method according to one further embodiment of the present invention.

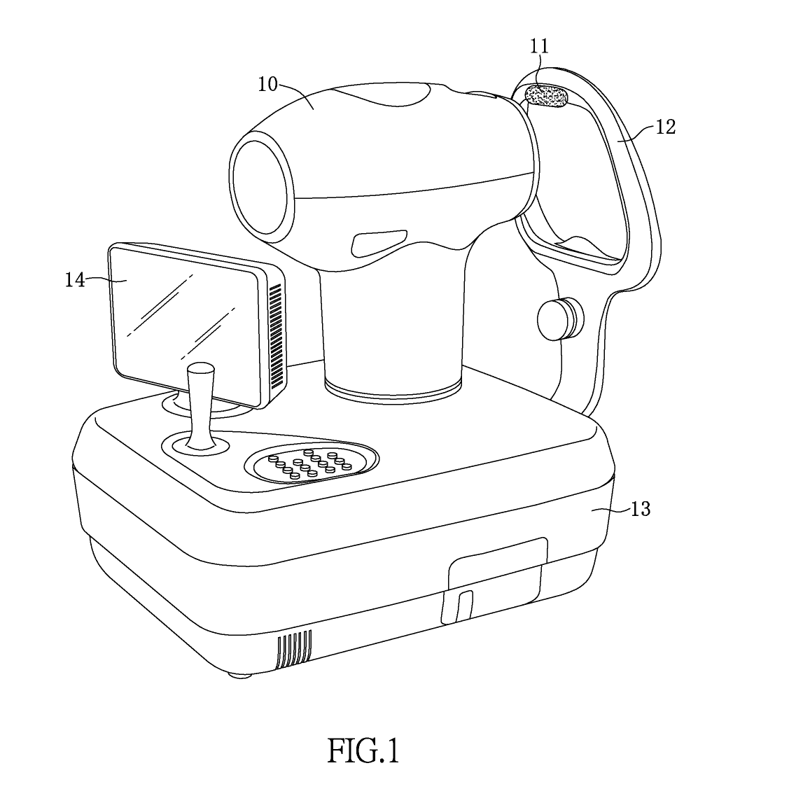

### DESCRIPTION OF THE EXEMPLARY EMBODIMENTS

Reference will now be made in detail to the exemplary embodiments of the present disclosure, examples of which are illustrated in the accompanying drawings. Wherever possible, the same reference numbers are used in the drawings and the description to refer to the same or like parts.

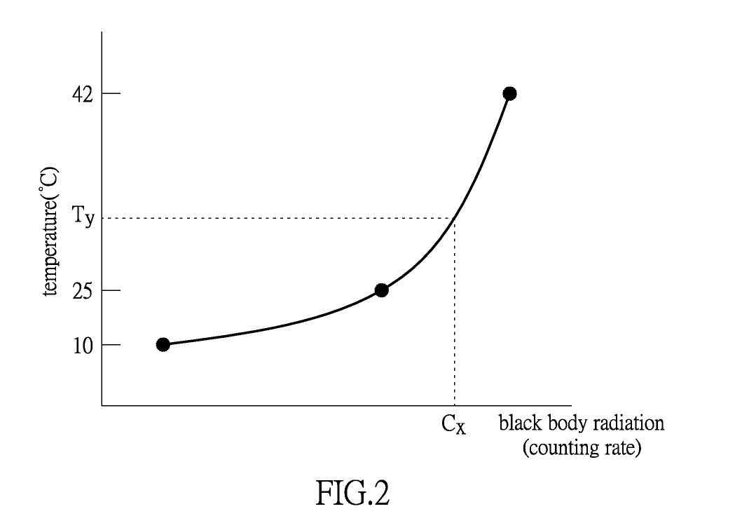

Thermography primarily indicates an infrared thermography (IRT). An infrared sensor installed in the thermography unit is used to image the infrared emitted from the object. The sensed temperature is compared with the temperature with respect to a black body radiation. The curve shown in FIG. 2 indicates a temperature-rising curve of the black body radiation. The temperature of the surface of an object can be measured by referring to the curve.

According to an embodiment of the present invention, the system for examining the function of eye gland may adopt the thermography. As shown in the diagram, the thermography includes an infrared thermal imaging sensor 10. A carrier frame body 12 is disposed in the front-end of the thermography unit. The carrier frame body 12 includes a ring structure extending upward from a base 13. The ring structure is used to carry the head of the human test subject. A black body 11 is disposed on the carrier frame body 12. When the thermography 10 measures the temperatures over the skin of the eyeball or eyelid of the human test subject, the thermography unit 10 simultaneously refers to the thermal radiation emitted from the blackbody 11. Therefore, the system can acquire the actual temperature over the skin of the eyeball or the eyelid.

In an exemplary example, the thermal imaging sensor 10 of the thermography unit acquires the thermal radiation for the blackbody 11. FIG. 2 shows a black body radiation value Cx which is employed to obtain a reference temperature Ty for calibration. Thus, the thermography unit starts detecting the radiation values at some specific positions of the skin of eyeball of the tested human. The computation circuit (not shown) of the thermography unit may be used to calculate the temperature corresponding to a radiation value from the temperature-rising curve. The temperature-rising curve for the blackbody defines the relationship between the temperature and the blackbody radiation value, e.g. between 10-degree Celsius to 42-degree Celsius. It is noted that the curve between 10-degree Celsius and 25-degree Celsius appears to have a linear relationship for the blackbody radiation; and the curve in the diagram between 25-degreen Celsius and 42-degree Celsius appears to have a quadratic relationship for the blackbody radiation. The relationship drawn by the curve may be introduced to the reference temperature Ty. A comparison table such as a lookup table may be used to find out the accurate temperature of the organ of the human test subject.

The thermography unit includes an infrared sensor inside the thermal imaging sensor 10. The infrared sensor is such as a thermopile, pyroelectric, or bolometer. After calibration referring to the blackbody radiation, skin temperature of the eyelid of the human test subject can be obtained according to the temperature-rising curve of the blackbody. The computation circuit 14 of the thermography unit is able to compute the average value for some specified positions. Further, the temperature differences among the specified positions may define indexes for diagnosing the ocular disease.

The system for examining functioning of the eye gland incorporates a contact/non-contact method to acquire temperature distribution on the eyelid. The temperature distribution may be used to check if any functional disturbance of the eye gland is found. The eye gland is such as lachrymal gland. One of the major functions of the eye gland is to excrete the oil layer of tear. If the eye gland meets with disturbance, the oil layer will have lower quality and result in eye inflammation. Thus the condition of the eye gland may be used to be a judgment indicator for diagnosing dry eye.

Further, the quality of oil layer excreted by the eye gland may be judged according to the temperature distribution and change over the skin of the eye. It is noted that the change of temperature is such as the characteristics of cooling. For example, inflammation may be developed if the temperature of eye increases. The system for examining the functions of the eye gland may adopt a non-contact method to acquire skin temperature of the eyeball. The non-contact method would not cause an uncomfortable problem like palpation.

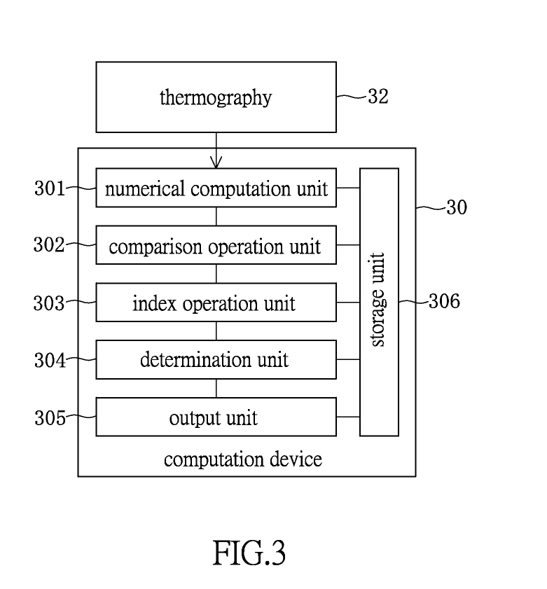

Reference is made to FIG. 3 depicting the hardware circuit modules or software modules for implementing the examining system according to one embodiment of the present invention.

In the diagram, the front-end of the system has a non-contact thermography unit 32 which is able to measure the skin temperature of an organ of a human test subject. The organ to be tested is such as the eye. The back-end of the system includes a computation device 30 which is used to process the temperature values obtained by the thermography unit 32. The computation device 30 may be an electronic device with computation capability. The computation device 30 may be integrated with the thermography unit 32 into one system. The computation device 30 and the thermography unit 32 may also be two standalone devices, and with a connectivity there-between. The connectivity made to link the two separate devices is such as a connection structure, cable, or over a specific communication protocol. The computation device 30 may be constructed by circuit modules or software modules.

The computation device 30 at least includes interconnected a numerical computation unit 301, a comparison operation unit 302, an index operation unit 303, a determination unit 304, an output unit 305, and storage unit 306. The storage unit 306 may implement the system memory, or data buffer for the circuit or software modules.

The numerical computation unit 301 is such as a computation unit for the computation device 30. One of the major functions of the numerical computation unit 301 is to compute the temperature distribution over the surface of the eyeball from the data measured by the thermography unit 32, especially to obtain temperature(s) within the range of one or more specific positions. After calibration referring to the black body radiation and related computation, a mean temperature among the positions can be obtained. The mentioned temperature information for every position, mean temperature or other data may be stored in the storage unit 306.

The comparison operation unit 302 retrieves temperature information from the storage unit 306. For the purpose of examining the function of the eye gland, the relationship of temperatures among the positions on the eye gland may be firstly obtained. For example, the temperature differences or ratios among the positions may be computed for the purpose of generating the indexes for diagnosis. The index operation unit 303 is then calculating using the judgment indexes for diagnosis of the eye gland based on the outputs of the comparison operation unit 302. The judgment indexes may be based on research of an ophthalmologist or medical institutes, and reflect the correlation of the function of the eye gland and temperature differences or ratios. In an exemplary embodiment, a lookup table is created. The lookup table may record the comparison between the temperature differences and the damage of an eye gland.

After that, the determination unit 304 of the computation device 30 is used to determine if any disorder of the eye gland is found or the disorder degree according to the judgment indexes. Then the output unit 305 outputs the judgment result. The outputs may be stored to the storage unit 306 which may be used to be system memory or the data buffer. The result outputted by the output unit 305 at least includes average value of the multiple temperature differences or ratios. The lookup table is created for recording the correlation of the function indexes of an eye gland and the average values.

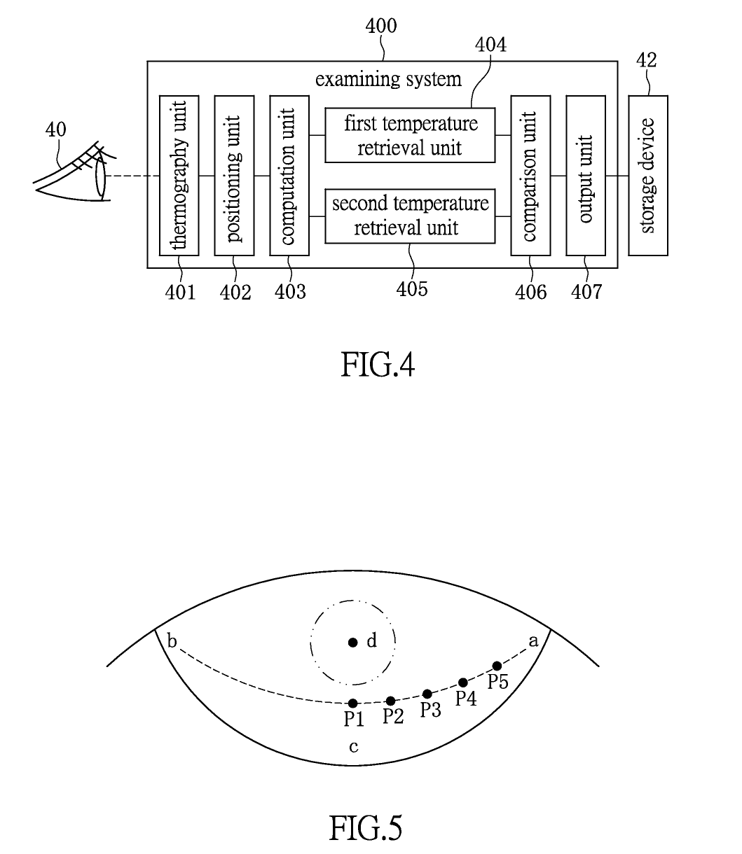

FIG. 4 shows another embodiment of the present invention. The integration of thermography and computation circuits or software modules embodying the examining system is shown.

The tested portion 40 indicates an eye shown in the diagram. One of the objectives of the examining system 400 is to measure the temperatures on the skin of eye, not only including the temperature of the ocular surface, but also the partial temperature of the cornea or conjunctiva. The examining system 400 is then used to measure the temperature change of the cornea or conjunctiva, and judge the condition of the eye gland.

The examining system 400 may be divided into multiple hardware or software modules according to its objectives and functions. The examining system 400 includes a thermography unit 401 which is used to sense and form a thermographic image of the tested portion 40. The thermography unit 401 may continuously capture a plurality of thermographic images within a period of time. After that, the thermography unit 401 may measure a temperature distribution among a plurality of positions in the thermographic image. The thermography unit 401 may be an independently-operating thermography unit, which may be referred to in FIG. 3.

In one embodiment, the system includes a positioning unit 402 coupled with the thermography unit 401. The positioning unit 402, through a software method, is used to position the plurality of preset positions over the ocular thermographic image. For example, the plurality of preset positions are on a certain range of the eyelid, and such as the shown tested positions along a line from left to right on the eyelid. Then the temperature distribution is obtained among the positions. A mean temperature within the certain range is calculated. The system is able to acquire the temperature distribution among the positions within the range in the thermographic image.

The computation unit 403 is used to compute the mean temperature from the at least one reference temperature of a reference position and a plurality of temperatures of the tested positions. The system includes memory for buffering the temperature values. In which, the reference temperature and the temperatures of the plurality of tested positions are respectively outputted to a first temperature retrieval unit 404 and a second temperature retrieval unit 405. The temperatures continuously acquired within a period of time are used to get an average value. The comparison unit 406 acquires the temperature values recorded in both the first temperature retrieval unit 404 and the second temperature retrieval unit 405. The reference temperature is allowed to compute the temperature differences or ratios from the temperatures of tested positions.

The output unit 407 is used to output the result from the comparison unit 406, and to the storage device 42. A final report may be produced.

FIG. 5 shows a schematic diagram depicting the tested portions for acquiring temperatures by the examining system in one embodiment of the present invention.

An appearance of an eyelid is schematically shown. The examining system utilizes a thermography unit or the like to capture a thermographic image of the closed eye. From the thermographic image, a temperature distribution can be formed, and a mean temperature of the temperatures of several areas of the eyelid are obtained.

The several tested positions are exemplarily specified from the rightmost position “a”, where is near the nose, to the leftmost position “b” on the eyelid. Then the temperatures of the tested positions are measured. In an exemplary embodiment, there are five tested positions P1, P2, P3, P4, and P5 which are uniformly defined between the position “a” and the central position “c” along the line from “a” to “b”. The mean temperature at every tested position (P1, P2, P3, P4, and P5) may cover a certain area, e.g. 1 to 3 mm. The examining system simultaneously acquires another mean temperature at the position “d” on the upper skin of the eyelid. After that, the temperature ratio or difference between every tested position (P1, P2, P3, P4, and P5) and the position “d” at the upper skin of eyelid can be computed. The relative relationship between the tested position “d” and every tested position forms the index to judge the condition of the eye gland. Therefore, for example, the inflammation index for the eye gland may be obtained.

According to one of the embodiments in the disclosure, the mentioned position “d” may be the position 1 cm extended from the position “c”. The temperature difference (Pi-“d”, i=1, 2, 3, 4, 5) or ratio (Pi/“d”, i=1, 2, 3, 4, 5) of every mentioned tested position P1, P2, P3, P4, or P5 and the tested position “d” is regarded as the inflammation index to diagnose the condition of the eye gland. The inflammation judgment based on the ocular inflammation index is: the inflammation occurs when the ratio is larger than 1.2, or the difference is larger 4.6 degrees Celsius; otherwise, the eye gland is healthy if lower or equal to the index.

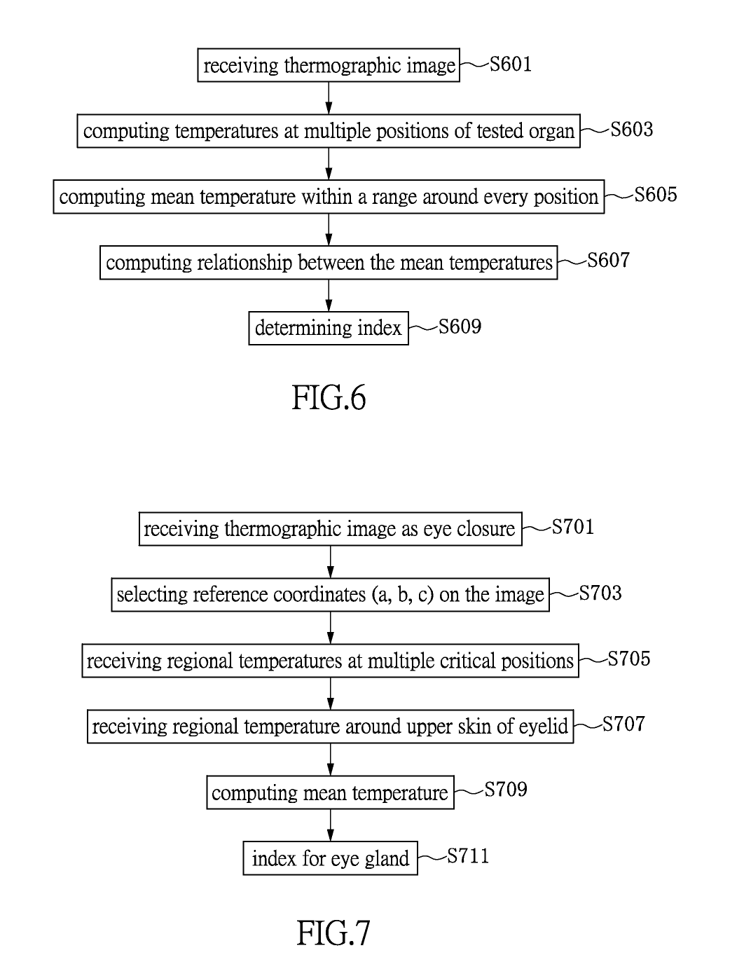

FIG. 6 shows a flow chart illustrating the method for examining the condition of an eye gland, and which is implemented by the examining system.

In the beginning, such as step S601, the examining system acquires a thermographic image of the tested organ by the thermography unit or thermography in the system. For example, the system acquires the thermographic image of the closed eye. Next, in step S603, through a positioning method, the system computes the temperatures at multiple positions of the tested organ. The related information includes the temperature distribution of the multiple positions, e.g. the multiple preset positions on the eyelid such as one reference position and several tested positions.

Next, in the step S605, an average value of the positions is computed. The average value represents the temperatures of the tested positions. In step S607, the system computes the correlation among average values, e.g. the differences or ratios between the reference temperature of reference position and the temperatures of tested positions. The temperature differences or ratios are references to define a threshold to judge the ocular function disturbance. Such as in step S609, it is used to judge the ocular inflammation.

It is noted that multiple temperature differences or ratios will be generated as referring to the temperatures at the mentioned multiple tested positions and the reference temperature, and therefore the average value among them may be employed to define an ocular inflammation index. For diagnosing the ocular inflammation in the preferred application, a lookup table may be created to render the correlation between the functional indexes of an eye gland and the temperature differences or the ratios.

Reference is made to FIG. 7, a flow chart is shown to depict the method for examining the function of an eye gland in accordance with the present invention.

In the beginning, as shown in step S701, the thermography or other like sensor circuit is used to capture a thermographic image of a closed eye. Next, as in step S703, a user may manipulate a software-initiated user interface for the examining system. Through the user interface, the user may review the image and choose reference coordinates for the thermographic image, e.g. the positions “a”, “b”, “c”, and the reference position “d” described in FIG. 5.

Next, such as in step S705, the system thereby acquires the regional temperatures around the multiple critical positions, such as, but not limited to, the positions “a”, “b”, “c”, and “d”. In step S707, a regional temperature on the upper skin of the eyelid, e.g. position “d”, is measured. After that, such as in step S709, an average value for those preset positions is computed. The temperature differences or ratios among those regional temperatures of the preset positions are acquired, so as to render the function indexes for the eye gland, such as in step S711.

Thus, the examining system for the eye gland function is disclosed for rendering the indexes to diagnose the condition of an eye gland in one preferred embodiment. It is appreciated that the ocular thermographic image is introduced to make the diagnosis through a non-contact examining method, for example to the dry eye. More specifically, several positions are designated on the eyelid. The temperature differences or ratios are later generated for judging the condition of the eye gland, especially to the quality of the oil layer of a tear that directly relates to the dry eye.

The above-mentioned descriptions represent merely the exemplary embodiment of the present disclosure, without any intention to limit the scope of the present disclosure thereto. Various equivalent changes, alterations or modifications based on the claims of the present disclosure are all consequently viewed as being embraced by the scope of the present disclosure.

What is claimed is:

1. An examining system for eye gland function, comprising:
a thermography unit, used to receive an ocular thermographic image, and a temperature distribution of multiple positions from the ocular thermographic image;
a computation unit, used to compute a mean temperature of the multiple positions within a range, including receiving a reference temperature of a reference position and temperatures of multiple tested positions, and the temperatures are stored in a memory of the examining system;
a comparison unit, calculating temperature differences or ratios among the reference temperature and the temperatures at multiple tested positions for acquiring the reference temperature and the temperatures at the tested positions; and
an output unit, outputting a result obtained by the comparison unit.

2. The examining system of claim 1, further comprising a positioning unit coupled with the thermography unit, used to position the multiple positions on the ocular thermographic image.

3. The examining system of claim 2, wherein, a mean temperature over an area of upper skin of an eyelid and multiple temperatures at the tested positions from left to right on the eyelid are obtained.

4. The examining system of claim 3, wherein the reference temperature at the reference position and the temperatures at the multiple tested positions are temporarily buffered to a first temperature retrieval unit and a second temperature retrieval unit of the system.

5. The examining system of claim 1, wherein the output result from the output unit at least includes an average value of the multiple temperature differences, or the ratios.

6. The examining system of claim 5, wherein, providing a lookup table for recording the average value of the multiple temperature differences or ratios, and correlation between the average value and a function index of an eye gland.

7. The examining system of claim 6, wherein the output result is stored in a storage unit.

8. The examining system of claim 1, wherein the thermography unit is an independently-operating thermography unit.

9. The examining system of claim 8, wherein the thermography unit includes an infrared thermography unit, a front end of the infrared thermography unit is disposed with a carrier frame body, and a black body is disposed on the carrier frame body.
